(19) United States
(12) Patent Application Publication
(10) Pub. No.: US 2005/0228498 A1
(43) Pub. Date: Oct. 13, 2005

(54) Osteoconductive integrated spinal cage and method of making same

(76) Inventor: Todd Andres, Evergreen, CO (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/991,956

(22) Filed: Nov. 18, 2004

Related U.S. Application Data
(60) Provisional application No. 60/523,288, filed on Nov. 18, 2003.

(51) Int. Cl. A61F 2/44; A61F 2/28

(52) U.S. Cl. 623/17.11; 623/23.51; 264/445

(57) ABSTRACT

The spinal cage comprises a structural component having sufficient strength to withstand the compressive loading between vertebral bodies. The structural component is integrated with an osteoconductive component to facilitate bone growth between the vertebral bodies. The structural component may comprise any of PEEK, PEKK, or other structural material. The osteoconductive component may comprise any of allograft, natural bone, tricalcium phosphate, hydroxyapatite or a blend of calcium carbonate, calcium lactate and other calcium salts. A method for making the spinal cage involves molding polymers around an osteoconductive component, heat staking, and may further include ultrasonically welding, snap fit or mechanically assembling and/or adhesively bonding components.

### RELATED APPLICATION DATA

This application claims priority under 35 U.S.C. § 119(e) from provisional application Ser. No. 60/523,288 filed Nov. 18, 2003, the disclosure of which is incorporated in its entirety herein by reference.

### BACKGROUND OF THE INVENTION

The present invention is directed to a spinal cage for use in spine surgery, and more particularly, is directed to an osteoconductive integrated spinal cage that may be made from a variety of materials, including PEKK, PEEK, porous hydroxyapatite, etc.

Back or spinal musculoskeletal impairments represent just over half of reported musculoskeletal impairments in the US. Additionally back and spinal musculoskeletal impairments are the leading cause of lost work productivity in the US according to a 1999 study of the American Academy of Orthopaedic Surgeons. According to the same study, 4.4 million people report inter-vertebral disc problems in the US. Although most patients recover using non-surgical therapies, many require surgical intervention to improve mobility and reduce pain. A common procedure used to address conditions such as degenerative disc disease, stenosis, and spondylolysis is referred to as spinal fusion. Approximately 350,000 spinal fusion surgical procedures are performed in the US each year.

Common spinal fusion procedures involve a discectomy (removal of the affected disc), fixation using metallic screws and rods, and replacement of the disc with a spinal cage to maintain proper vertebral spacing. Spinal cages, implanted during spinal fusion surgery, have been used for many years to restore and maintain disc height. Following removal of the defective disc, bone graft must be inserted into and around the spinal cage during the surgical procedure in order to facilitate fusion of the adjacent vertebral bodies. The bone harvest procedure is generally called iliac crest bone harvest, and it often results in donor site morbidity. According to a study published in the Journal Spine, 50.7% of patients who underwent the bone graft procedure experienced a significant morbidity such as ambulation difficulty, extending antibiotic usage, persistent drainage, and other problems.

Bone graft substitutes are now employed to eliminate the iliac crest bone harvest. However, significant preparation of the spinal fusion cage is required by the surgeon during the procedure. Bone graft substitutes and osteoconductive materials do not generally have sufficient mechanical properties needed to survive the compressive loading following spinal fusion surgery prior to the fusion between the vertebrae.

Prior art teaches the use of a spinal cage and with patient's own bone that is harvested during surgery and placed into and around the spinal cage. Other prior art utilizes bone graft substitutes or allograft bone (tissue bank derived) that is available as chips or granules and is placed into the spinal cage and around the spinal cage between the vertebral bodies. Still other prior art utilizes a collagen sponge that is soaked with blood, bone marrow, or bone morphogenic protein and placed into the spinal cage during surgery.

This invention also has application in other orthopedic applications including vertebral body replacements, trauma, hip revision surgery (cement restrictors) and others. In the case of trauma, this device can be used to provide osteointegration and mechanical support.

### SUMMARY OF THE INVENTION

There is provided in accordance with one aspect of the present invention, a method of making a spinal fusion implant. The method comprises the steps of positioning a structural component in contact with a porous osteoconductive component, under conditions such that surface material on the structural component flows into pores on the osteoconductive component and hardens, thereby providing an interlocking interface between the structural component and the osteoconductive component to produce a spinal fusion implant.

The conditions may include the application of heat, the application of ultrasound, the application of a solvent, or other techniques for generating a flowable material.

In accordance with a further aspect of the present invention, there is provided a method of making a spinal fusion implant. The method comprises the steps of providing a structural component dimensioned to fit within a disc space between two vertebral bodies, the structural component having a longitudinal axis, a transverse axis, a softening point and at least one channel extending generally parallel to the longitudinal axis. A porous osteoconductive component is provided. The osteoconductive component is heated to at least as high as the softening point for a surface on the structural component, and the osteoconductive component is forced into the channel to produce a spinal fusion implant.

In accordance with a further aspect of the present invention, there is provided a method of making a spinal fusion implant. The method comprises the steps of providing a structural component dimensioned to fit within a disc space between two vertebral bodies. The structural component has a longitudinal axis, a transverse axis, and at least one channel extending generally parallel to the longitudinal axis. At least a first transverse engagement surface is exposed to the channel.

A porous osteoconductive component is provided, having at least a second transverse engagement surface. The osteoconductive component is advanced into the channel such that the first engagement surface interlocks with the second engagement surface to retain the osteoconductive component within the structural component to produce a spinal fusion implant.

The second engagement surface may be carried by a radially outwardly extending support on the osteoconductive component. The support may comprise an annular ridge, such as a helical thread. Alternatively, the second engagement surface may comprise a portion of a wall defining a recess, such as a cavity, aperture, or radially inwardly extending annular recess.

In accordance with another aspect of the present invention, there is provided a device for placement between osseous structures. The device comprises a structural component having a first bone contacting surface spaced apart from a second bone contacting surface and a longitudinal axis extending therethrough. The structural component has sufficient strength along the axis to maintain spacing among the osseous structures, and the structural component is integrated with an osteoconductive component extending from the first face to the second face, to facilitate bone growth between the osseous structures.

The structural component may comprise a polymer, such as PEEK, PEKK, PEK, PEEKK, PEKEKK, or others known in the art.

The osteoconductive component may comprise at least one material selected from the group consisting of tricalcium phosphate, hydroxapatite, resorbable polymer, calcium filled resorbable polymer, calcium sulfate, allograft, and blends of any of these materials.

Preferably, the structural component comprises a polymer and has a compressive strength of greater than about 3,000 psi.

In accordance with a further aspect of the present invention, there is provided a spinal cage. The cage comprises a structural component having sufficient strength to withstand the compressive loading between vertebral bodies. The structural component is integrated across an engagement zone with an osteoconductive component that facilitates bone growth between the vertebral bodies.

It should be understood that this Summary of the Invention is not necessarily intended to encompass each and every aspect of the present invention and one of skill in the art will appreciate the full scope of the invention by the entire disclosure, including the claims, drawings, etc. as provided herein.

### BRIEF DESCRIPTION OF THE DRAWINGS

FIG. 1 is a schematic illustration of one embodiment of the present invention illustrating how the components of the present system are structurally related and combined by heat staking.

FIGS. 2A-2E are cross sectional schematic views of an assembled implant, illustrating various integration zones between the components in accordance with the present invention.

FIG. 3 is an illustration demonstrating one embodiment of the present invention in which an implant is made by over-molding a structural member onto an osteoconductive component.

FIG. 4 illustrates another embodiment of the present invention where ultrasonic welding is employed to make a vertebral body replacement component.

### DETAILED DESCRIPTION OF THE INVENTION

A spinal cage in accordance with the present invention comprises a structural component having sufficient column strength to withstand the axially compressive loading between vertebral bodies with such structural component integrated with an osteoconductive component to facilitate bone growth between the vertebral bodies. The structural component may comprise any of a variety of polymers or metals such as titanium, titanium alloys or stainless steel, and preferably comprises PEEK, PEKK, and others known in the art. In a preferred embodiment, an osteoconductive component is intimately bonded to or secured to the structural component, to produce an integral spinal cage. The osteoconductive component may be resorbable, and may comprise allograft, natural bone, tricalcium phosphate, hydroxyapatite or a blend of calcium carbonate, calcium lactate and other calcium salts.

In one embodiment, the spinal cage is manufactured with an osteoconductive material within or around the spinal cage. The osteoconductive spinal cage integrates the spinal cage and the bone graft substitute in a single, easy to handle device. By preloading the spinal fusion cages using these techniques, surgery duration and patient morbidity may be reduced. The present invention preferably involves the use of an osteoconductive material that is substantially more rigid than collagen. One embodiment of the device may use HA or other rigid osteoconductive substances with a reasonable shelf life (e.g., shelf life in excess of about one year) that accomplish the objective of providing a structure conducive to bone growth. Included substances are various forms and compounds of bioabsorbable polymers, various forms of calcium compounds, etc.

In one preferred embodiment, the device comprises a PEKK or PEEK-type structural polymer incorporating a rigid osteoconductive substance into a spinal fusion cage using manufacturing techniques such as over molding, heat staking, or ultrasonic welding. One preferred embodiment of the osteoconductive material is porous hydroxyapatite (HA). The HA component may be essentially flush with or protrude slightly from the bottom and top surfaces in the interior portion of the cage where the fusion is designed to occur. The HA component is mechanically or thermo-mechanically lodged within the interior of the cage. The polymer may infuse into the outer surface of the porous HA.

Several options exist for incorporating the HA component within the interior body of the polymer cage. If a non-porous HA or other osteoconductive component, such as a bioabsorbable polymer is used, then the osteoconductive component can be designed to create a mechanical interlock with the polymer spinal cage (outer portion). Methods of incorporating the osteoconductive material into the polymer cage to produce an integrated product include:

1. Insert/over mold the osteoconductive component within the cage;

2. Heat stake the osteoconductive component into the cage (would work with extruded/machined cages);

3. Ultrasonically press the osteoconductive component into the cage (OK for extruded/machined cages); and/or

4. Use an osteoconductive portion and polymer structural component machined such that the two fit together with a snap fit or interlocking mechanism or keyed mechanism.

Alternatively, one or both of the structural component and osteoconductive component can be machined with complementary surface structures such that there is a mechanical interference when the two are assembled, keeping them intact relative to one another.

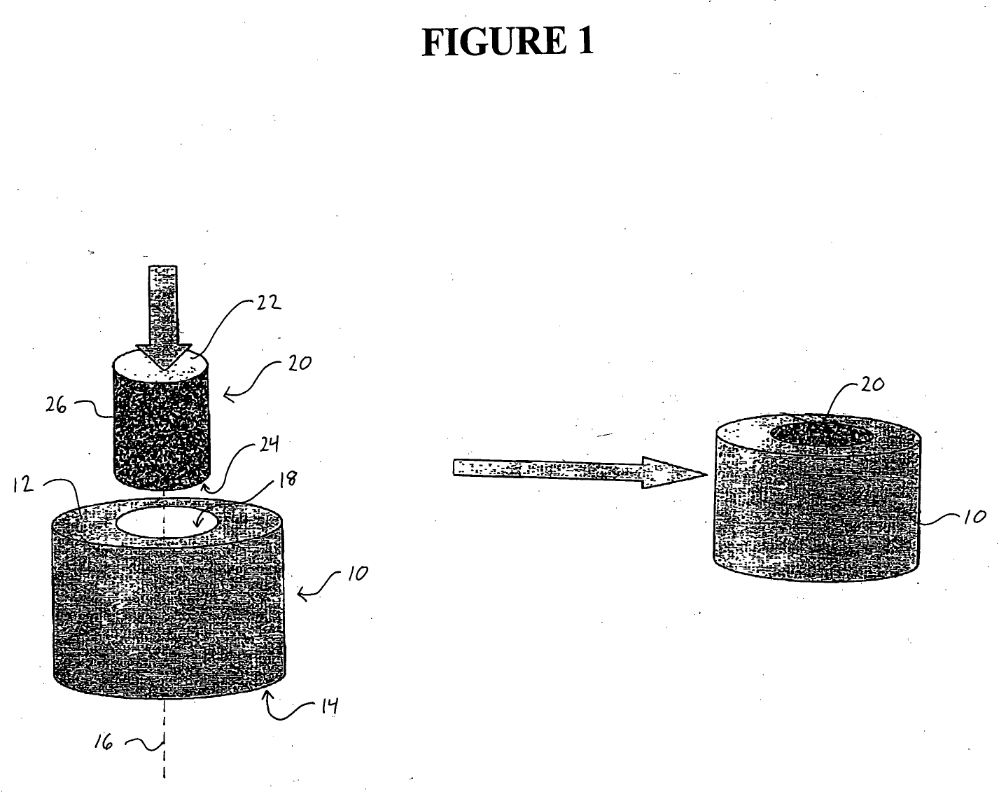

Referring to FIG. 1, there is illustrated in schematic fashion a structural member 10 in accordance with one aspect of the present invention. The structural member 10 has a first surface 12 which may in use be a superior surface for mounting in contact with a superior vertebral body. A second surface 14 opposes the first surface 12, and it may in use be positioned against an inferior vertebral body. The first surface 12 and second surface 14 are separated by the axial length or height of the structural member 10, which will vary depending upon the intended use of the implant. In general, the axial height may range from approximately 4 mm to approximately 150 mm or more, depending upon the intended use. In one particular application, the structural member 10 is configured for implantation within a disc space in-between a forth and fifth lumbar vertebrae. For this use, in a human adult, the axial length of the structural member 10 between the first surface 12 and second surface 14, will be within the range of from about 6 mm to about 14 mm. For use as a replacement for the L-5 vertebral body, for example, the structural member will have an axial length of about 70 mm.

Structural member 10 may be described with reference to a longitudinal axis 16, which extends between the first surface 12 and second surface 14. Although the structural member 10 in the illustrated embodiment exhibits radial symmetry about the longitudinal axis 16, that may not be necessary depending upon the desired clinical performance of the implant. Viewed along the longitudinal axis 16, the structural member 10 may have any of a variety of non-regular geometric shapes, such as oval, circular with a flat side, or kidney bean shape such that a first side is concave towards the longitudinal axis 16 and a second opposing side is convex toward the longitudinal axis 16. Shapes which are more distant from the normal anatomy may also be utilized, such as square, rectangular, hexagonal, or other geometric shape.

The illustrated embodiment takes the form of a generally cylindrical configuration, having a circular cross section in a plane transverse to the longitudinal axis 16. The outside diameter of the structural member 10 may be varied depending upon the particular anatomy into which the implant is to be placed, but in general will range from about 8 mm to about 35 mm. For this purpose, the term “diameter” refers to the true diameter where the implant has a circular configuration or the diameter of the smallest circle which can enclose an implant having a noncircular configuration: In general, spinal cages in accordance with the present invention intended for implantation within the cervical spine will have an outside diameter of no greater than about 20 mm. Spinal cages intended for implantation in the lumbar spine will have an outside diameter of no greater than about 35 mm. In use, either a single implant may be positioned at the implant site, or 2 or 3 or 4 or more smaller implants may be positioned side by side at the implantation site.

The structural member 10 may be formed in any of a variety of ways, depending upon the materials used. For example, it can be machined from a solid block of material, molded such as injection molded or otherwise formed in its final shape, as will be understood by those of skill in the art.

The basic polymers of interest for use as the structural component include aromatic polyketones such as polyetheretherketone (PEEK), polyetherketoneketone (PEKK), polyetherketonetherketoneketone (PEKEKK), polyetheretherketoneketone (PEEKK), and polyetherketone (PEK). Other polymer materials may be used, preferably being biocompatible and resistant to lipids. Aromatic polyketones have a melting temperature around 644 to 720 degrees F.

The structural member 10 is additionally provided with at least one cavity or channel 18 extending axially therethrough. In the illustrated embodiment, the channel 18 is approximately concentrically oriented with respect to the longitudinal axis 16. The longitudinal axis of the channel 18 may be offset laterally from the longitudinal axis 16 of the structural member 10, depending upon the desired configuration and performance of the device.

Channel 18 may be provided in any of a variety of ways, such as by an insert in the molding process, or by post formation drilling using mechanical drills or other techniques. Channel 18 extends between a first opening on the first surface 12 of the structural member 10 and a second opening on the second surface 14 of structural member 10, to enable communication throughout the axial length of the structural member 10.

At least one osteoconductive portion 20 is configured for positioning within the channel 18. In the illustrated embodiment, osteoconductive portion 20 has a generally cylindrical configuration, for corresponding with the configuration of the channel 18. Osteoconductive portion 20 extends between a first face 22 and a second face 24, defining a side wall 26 extending therebetween.

In accordance with the heat staking embodiment of the present invention, osteoconductive portion 20 is provided with an exterior configuration which corresponds approximately to the interior configuration of the channel 18. Preferably, in a cylindrical embodiment as illustrated, the diameter of the osteoconductive portion 20 is at least about 0.5%, often at least about 1%, and in certain embodiments at least about 2½% or 5% greater than the inside diameter of the channel 18. As such, the osteoconductive portion 20 will not fit within channel 18 without either compression of the osteoconductive portion 20 or the expansion of the channel 18.

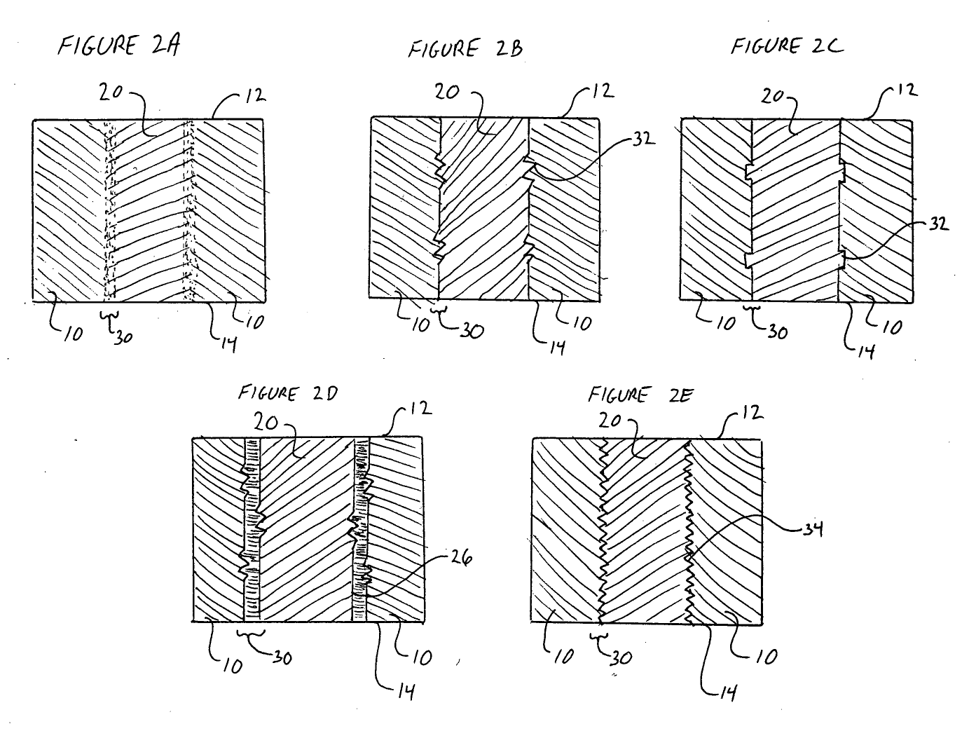

For certain preferred construction materials of the orthopedic implant described herein, the osteoconductive portion 20 will withstand significantly greater temperatures than the structural member 10, before softening occurs. Under these conditions, the osteoconductive portion 20 may be heated to a temperature which is at least as high as the softening point for the material of the inside surface of the structural member 10, and preferably is above the melting point of the material of the inside surface of structural member 10. The heated osteoconductive portion 20 may thereafter be press fit along the longitudinal axis 16 into the channel 18. As the osteoconductive portion 20 contacts the structural member 10, a surface layer lining the channel 18 softens or melts, allowing the osteoconductive portion 20 to be force fit completely into the channel 18. Due to the porous configuration of the osteoconductive portion 20, small amounts of softened or melted material from structural member 10 flow into the porous side wall 26. Upon cooling, the material of structural member 10 solidifies, providing an integration zone (see FIG. 2) between the osteoconductive portion 20 and the structural member 10. Within this integration zone, material of the structural member 10 flows into the interstitial microporous or porous surface structure of the osteoconductive portion 20, and hardens thereby providing a secure interlocking fit between the two components.

Each of the osteoconductive portion 20 and structural member 10 may comprise a homogeneous material throughout. Alternatively, either the side wall 26 of the osteoconductive portion 20 or the surface of the structural member 10 which surrounds the channel 18 may be provided with a coating or layer such as a tie layer for facilitating bonding between the two components. Any of a variety of thermoplastic materials may be utilized for the tie layer such as polyethylene, and still permit manufacturing through the foregoing heat staking method.

Referring to FIGS. 2A-2E, there is illustrated a series of schematic cross sectional views through the assembled implant formed in accordance with the present invention. As illustrated, the structural component 10 encases the osteoconductive portion 20. A boundary or integration zone 30 is created between the two components, forming a positive mechanical interlock. Integration zone 30 is formed by the material of either the osteoconductive portion or the structural member or a tie layer flowing into pores or apertures or against other interference surfaces of the complementary component.

Referring to FIG. 2A, there is illustrated a schematic cross-sectional elevational view through an implant assembled using the process described in connection with FIG. 1. An integration zone 30 is formed where material of the structural member 12 has flowed into pores on the osteoconductive portion 20 and solidified, to form a bond.

The radial direction depth of the integration zone 30 will be a function of the viscosity of the softened polymer as well as the pore size into which the polymer may flow. In general, the depth of the boundary zone will be in the range of from about 100 microns to about 2 mm or more, and often at least about 0.1 mm, such as in the range of about 0.5 mm to 1 mm, in the case of a porous HA osteoconductive portion bonded to a PEEK structural component.

Any of a variety of additional structures may be provided, for enhancing the mechanical interlock within integration zone 30. For example, the osteoconductive portion 20 may be provided with one or more radially outwardly extending projections 32, to be received within one or more corresponding recesses on the wall defining channel 18 on the structural member 10. See FIGS. 2B and 2C. In one implementation of the invention, the radially outwardly extending projections 32 on osteoconductive portion 20 take the form of one or two or more annular flanges, which are adapted to fit within corresponding annular recesses extending radially outwardly from the channel 18 into the structural member 10. The apertures or annular recess extending radially outwardly from the channel 18 may be formed by softening the material of the structural member 12 and forcing the osteoconductive portion 20 therein as has been described.

In a variation illustrated in FIG. 2E, the side wall 26 of the osteoconductive portion 20 may be provided with a helical thread 34, which may be received within a corresponding helical thread on the wall of channel 18, or which may be press fit into the structural member 10 under heat and pressure as has been described.

The orientation of any of the foregoing structures can be reversed, such that the osteoconductive portion 20 is provided with one or more radially inwardly extending recesses, to receive projections from the structural member 10. In each of these configurations, at least one projection on either the osteoconductive portion 20 or the structural member 10 provides a surface which extends transversely to the longitudinal axis 16. That transverse surface cooperates with a complementary transverse surface on the other of the osteoconductive portion 20 or structural member 10 to provide a mechanical interfit, between the two, and resist axial movement of the osteoconductive portion 20 with respect to the structural member 10.

As a further alternative, the osteoconductive portion 20 may be secured within structural member 10 using any of a variety of adhesives or tie layers which may be caused to flow between the side wall 26 of osteoconductive portion 20 and the wall defining channel 18, with or without the provision of additional surface structures. See FIG. 2D. Solidifiable polymers such as polymethylmethacrylate (PMMA) may be heated or mixed and caused to flow into the space between the two components of the implant. Adhesives such as Master Bond may also be used.

In the embodiment illustrated in FIG. 2D, each of the surface 26 of the osteoconductive portion 20 and the wall defining the channel 18 are provided with surface structures to provide a mechanical interfit with the solidified polymer, so that the integrity of the junction is not limited to the shear force of the bond between the hardenable media and the component parts. Depending upon the nature of the hardenable media, and the materials of the hardenable media and the components of the implant, the provision of surface structures may be included or omitted as will be apparent to those of skill in the art.

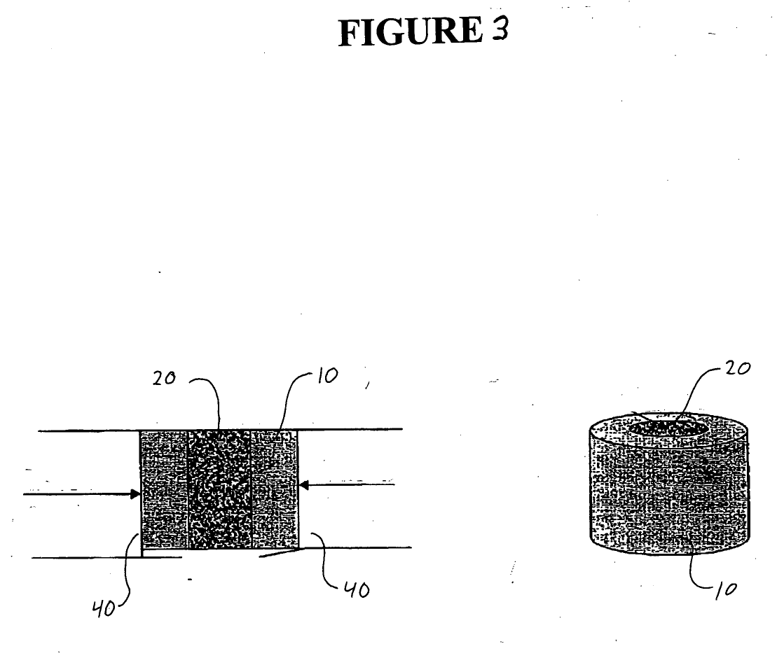

Referring to FIG. 3, there is illustrated an over molding process of manufacturing implants in accordance with the present invention. Additional details are provided in Example 2 below.

In general, a mold 40 is provided having a cavity therein which is configured to produce an implant having the desired exterior configuration. In the illustrated embodiment, the mold cavity has a substantially cylindrical configuration. An osteoconductive portion 20 is preformed, and positioned within the mold cavity to leave a space between the osteoconductive portion 20 and the surface of the mold cavity. In the illustrated embodiment, the osteoconductive portion is positioned generally coaxially with the longitudinal axis of the mold cavity, leaving a generally toiroidal space between the osteoconductive portion and the surface of the mold. In one embodiment, the osteoconductive portion comprises porous HA.

The structural member 10 is thereafter formed by injecting molten material into the remaining space in the mold cavity. The molten material is thereafter caused to solidify, such as by cooling, polymerization, or other process. In one implementation of the invention, the molten material comprises PEEK, which has been heated above its melting point.

The osteoconductive portion 20 may be preheated, prior to introduction of the molten PEEK, to optimize the depth of the integration zone 30. The integrity of the integration zone 30 may be enhanced in any of a variety of additional ways, as desired, such as by increasing the porosity of the surface of the osteoconductive portion 20, providing additional ridges, grooves, or surface textures on either or both of the osteoconductive portion 20 and the wall defining channel 18, described above.

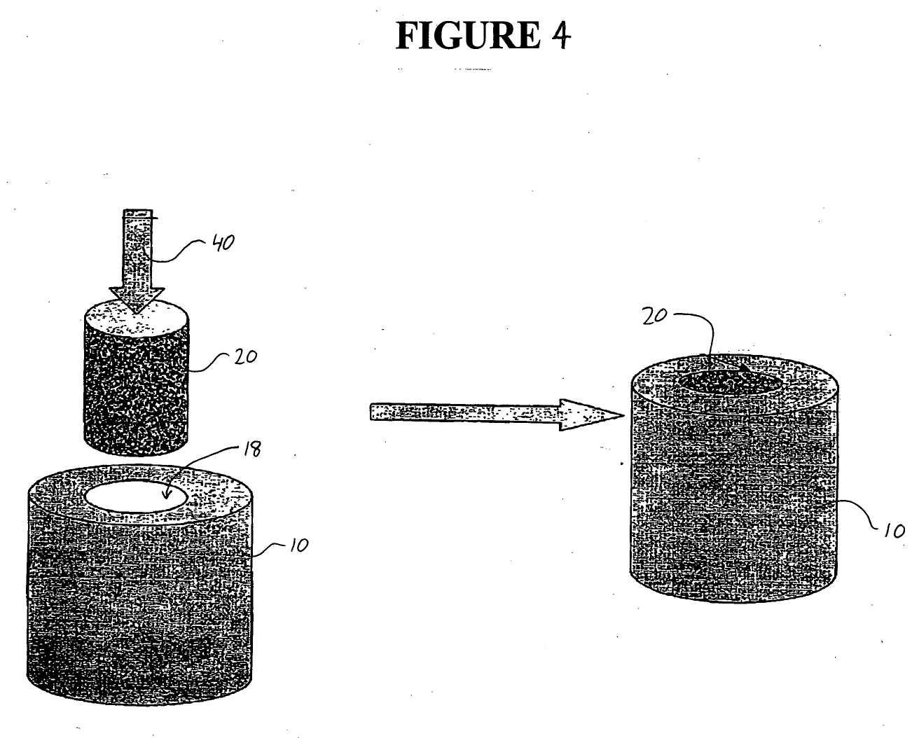

Referring to FIG. 4, there is schematically illustrated an alternative method of assembling the spinal cage in accordance with the present invention. In this embodiment, the osteoconductive portion 20 is dimensioned slightly larger than the channel 18 in a structural member 10. One or both of the osteoconductive portion 20 and structural member 10 is softenable upon application of high frequency ultrasonic energy. The osteoconductive portion 20 is aligned coaxially with the longitudinal axis extending through the channel 18. An ultrasonic transducer 40 is placed in ultrasonic transmission contact with the osteoconductive portion 20, and activated to couple the transducer to the osteoconductive portion 20. Force is applied along the longitudinal axis, to drive the osteoconductive portion 20 into the channel 18. Ultrasonic vibration of the osteoconductive portion 20 (e.g. porous HA) caused frictional heating of the inner wall of the structural member 10 (e.g. PEKK). The interior surface melts into the porous HA structure, causing a solid structural composite of PEKK and HA.

The benefits derived from the integrated assembly of the present invention involve timesavings for the surgeon and operating staff during the procedure. In prior art techniques, a surgeon may often compound a blend of crushed iliac crest bone and marrow, blood, or BMP for placement into the cage just prior to implantation. Handling of this mixture during implantation within the cage is cumbersome. Incorporating an “all in one” cage with an interior osteoconductive structure eliminates the additional iliac crest procedure. Prior to implantation of this cage, the surgeon only needs to soak or saturate (for effective periods of time) the HA cage interior with the patient's own marrow or blood, plasma rich platelets, and or Bone Morphogenic Protein. This eases handling and reduces the surgical procedure duration.

The osteoconductive portion is preferably machined to take up the space within the interior of the polymer structural component. The polymer structural component is designed such that it can be implanted between the vertebral bodies using the preferred technique of the surgeon and sized in a manner sufficient to stabilize the vertebral bodies and restore the proper disc height. If the polymer component is machined, it is preferably machined to proper dimensions such that it can mechanically maintain the disc space and allow for heat staking or ultrasonically welding of the osteoconductive portion. When using the osteoconductive portion as the interior of the device, the osteoconductive portion is designed with a slight interference fit within the structural component.

The following patents are incorporated herein by this reference in their entireties to provide background with respect to particular techniques that may be employed in practicing the present invention: U.S. Pat. No. 4,767,298 (with respect to heat staking); U.S. Pat. No. 3,666,602 (with respect to ultrasonic welding); and U.S. Pat. No. 4,075,820 (with respect to spin welding). One of skill in the art will appreciate from these references and the guidance provided herein how to make and use the various embodiments of the invention as set forth herein.

Further embodiments of the present invention, including both the device and the method for making such device, will be understood by one of skill in the art by referencing the below prophetic examples.

### PROPHETIC EXAMPLES

### Example 1

### Spinal Cage Made by Heat Staking of HA Osteoconductive Component into PEKK Structural Component

One of several manufacturing methods used to join the osteoconductive portion with the polymer structural component includes heat staking. In this example, the osteoconductive material is preferably composed of a substance(s) that can be heated to above the melting temperature of the polymer without significant degradation. Osteoconductive materials that may be used in this method include ceramics such as porous hydroxyapatite (HA) and calcium phosphate. The HA component must be slightly larger than the polymer component so that when inserted a slight interference fit is developed such that mechanical forces will prevent the two from separating. To use heat staking as the joining method, one must preheat the osteoconductive portion significantly above the melting temperature of the structural polymer component. For example, using PEKK polymer, one must preheat the HA osteoconductive component to a temperature above the softening point of the polymer, and preferably above the melting point of the polymer such as to above about 700° F. or about 730° F. and press the preformed HA component into the fixed PEKK component using a hydraulic press while the HA is preferably still significantly above 680° F. (the melting temperature of the PEKK polymer). As the HA component is forced into the polymer component, it causes the surfaces touching the HA component to melt. As the polymer melts, it flows slightly into the porous HA causing a mechanical interlock between the HA and PEKK polymer.

### Example 2

### Over Molding PEEK Polymer Over Tri-Calcium Phosphate

In this example, the PEEK polymer is the structural component of the spinal cage, and tri-calcium phosphate is the osteoconductive portion of the spinal cage. A mold designed to create the proper shape for intervertebral implantation, restoration of disc height, and over molding of the tri-calcium phosphate portion. In this case, the tri-calcium phosphate actually makes up the interior surfaces of the polymer mold. The tri-calcium phosphate is designed such that there is a mechanical locking caused between the polymer and tri-calcium phosphate (TCP). For example, the TCP component can be shaped such that it has small apertures or appendages that the polymer is formed into or around when it is melted over the surface. The preferred method for melting the polymer in a mold around the TCP is injection molding, however, compression molding may also work. This over molding process includes inserting the TCP into the mold and holding the TCP within the mold by the exposed sections (top and bottom) and causing molten PEEK resin to flow into the mold cavity. The interior of the mold cavity creates the exterior shape of the PEEK, and the TCP creates the interior surfaces of the PEEK. The PEEK shrinks onto the TCP creating a preload and the pre-designed mechanical interference.

### Example 3

### Snap Fit of Resorbable Polymer onto Titanium Spinal Cage

In this example, a resorbable polymer such as polyglycolic acid (PGA) is used to form the osteoconductive portion of the spinal cage, and titanium is used for the structural component. In this method, clinically superior results may be achieved if the resorbable polymer is porous and filled with a small amount of calcium sulfate. The resorbable polymer shape is designed such that it includes a flexible snap for incorporating with the titanium structural component. Substantial design freedom exists in this instance. In general, snap fits may be achieved by providing an extension on one of the two components which is received within a complementary recess on the other of the two components. The titanium can make up the interior, exterior, or even side portion of the cage in order to cause bone growth through out the inter-vertebral space.

### Example 4

### A Ultrasonically Welded HA/PEEK Cement Restrictor

In this example, a hip revision cement restrictor is manufactured using ultrasonic techniques. The exterior portion is PEEK polymer and is used to provide structural support while the interior portion is porous bioresorbable polymer PLLA. In this case, the PEEK exterior portion is held fixed while the PLLA is ultrasonically welded into the interior portion of the PEEK. The exterior surfaces of the PLLA are caused to melt by ultrasonic vibration. The PLLA then conforms to the PEEK portion and is fixed. Now the cement restrictor may be sterilized and implanted during hip revision surgery. The cement restrictor is used to prevent unwanted migration of bone cement.

While various embodiments of the present invention have been described in detail, it will be apparent that further modifications and adaptations of the invention will occur to those skilled in the art. It is to be expressly understood that such modifications and adaptations are within the spirit and scope of the present invention.

1. A device for placement between or among osseous structures comprising a structural component having a first bone contacting surface spaced apart from a second bone contacting surface and a longitudinal axis extending therethrough, the structural component having sufficient strength along the axis to maintain spacing among the osseous structures, and said structural component integrated with an osteoconductive component extending from the first face to the second face to facilitate bone growth between the osseous structures.

2. A device as in claim 1, wherein the structural component comprises titanium.

3. A device as in claim 1, wherein the structural component comprises a polymer.

4. A device as in claim 3, wherein the polymer is selected from the group consisting of PEEK, PEKK, PEK, PEEKK and PEKEKK.

5. A device as in claim 1 wherein the osteoconductive component comprises at least one material selected from the group consisting of porous tricalcium phosphate, hydroxyapatite, resorbable polymer, calcium filled resorbable polymer, calcium sulfate, allograft, and blends of any of these materials.

6. A device as in claim 5, wherein the material is porous.

7. A device as in claim 1, dimensioned for placement between a first and a second vertebral bodies.

8. A device as in claim 1, dimensioned for replacement of a vertebral body.

9. A spinal cage, comprising a structural component having sufficient strength to withstand the compressive loading between vertebral bodies, said structural component integrated across an engagement zone with an osteoconductive component that facilitates bone growth between the vertebral bodies.

10. The spinal cage of claim 9, wherein the structural component comprises a polymer and has a compressive strength greater than about 3000 psi and is biocompatible.

11. The spinal cage of claim 10, wherein the polymer is PEEK.

12. The spinal cage of claim 10, wherein the polymer is PEKK.

13. The spinal cage of claim 9, wherein the osteoconductive component comprises allograft or natural bone.

14. The spinal cage of claim 9, wherein the osteoconductive component is primarily composed of calcium.

15. The spinal cage of claim 14, wherein the osteoconductive component comprises tri-calcium phosphate.

16. The spinal cage of claim 15, wherein the osteoconductive component comprises porous tri-calcium phosphate.

17. The spinal cage of claim 9, wherein the osteoconductive component comprises hydroxyapatite.

18. The spinal cage of claim 17, wherein the osteoconductive component comprises porous hydroxyapatite.

19. The spinal cage of claim 14, wherein the osteoconductive component comprises a blend of calcium salts.

20. The spinal cage of claim 9, wherein the osteoconductive component comprises a blend including at least one of calcium carbonate and calcium lactate.

21. The spinal cage of claim 9, wherein the osteoconductive component comprises a resorbable polymer.

22. The spinal cage of claim 21, wherein the osteoconductive component comprises a porous resorbable polymer.

23. The spinal cage of claim 21, wherein the osteoconductive component comprises a calcium filled resorbable polymer.

24. The spinal cage of claim 9, wherein the osteoconductive component is heat staked into the structural component.

25. The spinal cage of claim 10, wherein the osteoconductive component is over molded by the polymer that makes up the polymer structural component using injection or compression molding into or around the osteoconductive component.

26. The spinal cage of claim 10, wherein the osteoconductive component is ultrasonically welded into the polymer structural component.

27. The spinal cage of claim 10, wherein the osteoconductive component is inserted into a preheated polymer structural component such that when the polymer structural component cools it decreases in size due to thermal contraction upon cooling creating a mechanical load on the osteoconductive component.

28. The spinal cage of claim 9, wherein the osteoconductive component comprises at least a first engagement surface which interlocks with at least a second, complementary engagement surface on the structural component.

29. The spinal cage of claim 9, wherein the osteoconductive component is adhesively bonded using a biocompatible adhesive, to the structural component.

30. The spinal cage of claim 9, wherein the structural component comprises biocompatible metal, such that:
a) the metallic structural component has sufficient strength to withstand the compressive loading within the vertebral bodies; and
b) an osteoconductive component is mechanically fixed within or around the metallic structural component.

31. The spinal cage of claim 9, wherein the structural component comprises biocompatible ceramic, such that the ceramic structural component has sufficient strength to withstand the compressive loading within the vertebral bodies; and
an osteoconductive component mechanically fixed within or around the ceramic structural component.

32. A method of making a spinal cage, comprising providing an osteoconductive component and a structural component, and incorporating said osteoconductive component and said structural component to produce said cage by heating said osteoconductive components, ultrasonically pressing said osteoconductive component onto said cage; or machining an osteoconductive portion, which comprises said osteoconductive component, so that said portion inter-locks with a polymer structural component.

33. A method of making a spinal fusion implant, comprising the steps of:
providing a structural component dimensioned to fit within a disc space between two vertebral bodies, the structural component having a longitudinal axis, a transverse axis, a softening point and at least one channel extending generally parallel to the longitudinal axis;
providing a porous osteoconductive component;
heating the osteoconductive component to at least as high as the softening point of the structural component; and
forcing the osteoconductive component into the channel to produce a spinal fusion implant.

34. A method of making a spinal fusion implant, comprising the steps of positioning a structural component in contact with a porous osteoconductive component under conditions such that surface material on the structural component flows into pores on the osteoconductive component and hardens, thereby providing an interlocking interface between the structural component and the osteoconductive component to produce a spinal fusion implant.

35. A method as in claim 34, wherein the conditions include the application of heat.

36. A method as in claim 34, wherein the conditions include the application of ultrasound.

37. A method as in claim 34, wherein the conditions include the application of a solvent.

38. A method of making a spinal fusion implant, comprising the steps of:
providing a structural component dimensioned to fit within a disc space between two vertebral bodies, the structural component having a longitudinal axis, a transverse axis, at least one channel extending generally parallel to the longitudinal axis, and at least a first transverse engagement surface exposed to the channel;
providing a porous osteoconductive component having at least a second transverse engagement surface;
advancing the osteoconductive component into the channel such that the first engagement surface interlocks with the second engagement surface to retain the osteoconductive component within the structural component to produce a spinal fusion implant.

39. A method of making a spinal fusion implant as in claim 38, wherein the second engagement surface is carried by a radially outwardly extending support.

40. A method of making a spinal fusion implant as in claim 39, wherein the support comprises an annular ridge.

41. A method of making a spinal fusion implant as in claim 39, wherein the support comprises a helical thread.

42. A method of making a spinal fusion implant as in claim 38, wherein the second engagement surface is a portion of a wall defining a recess.
